SEQUENCE LISTING (21 sequences)

SEQ ID NO: 1 - protein, 69 aa, Artificial sequence
  Synthetic peptide
GPYGANMEDSVCCRDYVRYRLPLRVVKHFYWTSDSCPRPGVVLLTFRDKEICADPRVPWVKMILNKLSQ

SEQ ID NO: 2 - protein, 69 aa, Artificial sequence; identical to SEQ ID NO: 1
  Synthetic peptide

SEQ ID NO: 3 - protein, 69 aa, Artificial sequence; identical to SEQ ID NO: 1
  Synthetic peptide

SEQ ID NO: 4 - protein, 69 aa, Artificial sequence; identical to SEQ ID NO: 1
  Synthetic peptide

SEQ ID NO: 5 - protein, 69 aa, Artificial sequence; identical to SEQ ID NO: 1
  Synthetic peptide

SEQ ID NO: 6 - protein, 69 aa, Artificial sequence; identical to SEQ ID NO: 1
  Synthetic peptide

SEQ ID NO: 7 - protein, 76 aa, Artificial sequence
  Synthetic peptide
QPDAINAPVTCCYNFTNRKISVQRLASYRRITSSKCPKEAVIFKTIVAKEICADPKQKWVQDSMDHLDKQTQTPKT

SEQ ID NO: 8 - protein, 74 aa, Artificial sequence
  Synthetic peptide
GPASVPTTCCFNLANRKIPLQRLESYRRITSGKCPQKAVIFKTKLAKDICADPKKKWVQDSMKYLDQKSPTPKP

SEQ ID NO: 9 - protein, 77 aa, Artificial sequence
  Synthetic peptide
GTNDAEDCCLSVTQKPIPGYIVRNFHYLLIKDGCRVPAVVFTTLRGRQLCAPPDQPWVERIIQRLQRTSAKMKRRSS

SEQ ID NO: 10 - protein, 68 aa, Artificial sequence
  Synthetic peptide
KPVSLSYRCPCRFFESHVARANVKHLKILNTPNCALQIVARLKNNNRQVCIDPKLKWIQEYLEKALXK

SEQ ID NO: 11 - protein, 74 aa, Artificial sequence
  Synthetic peptide
GPASVPTTCCFNLANRKIPLQRLESYRRITSGKCPQKAVIFKTKLAKDICADPKKKWVQDSMKYLDQKSPTPXP

SEQ ID NO: 12 - protein, 69 aa, Artificial sequence
  Synthetic peptide
GPYGANMEDSVCCRDYVRYRLPLRVVKHFYWTSDSCPRPGVVLLTFRDKEICADPRVPWVKMILNXLSQ

SEQ ID NO: 13 - protein, 72 aa, Artificial sequence
  Synthetic peptide
SAKELRCQCIKTYSKPFHPKFIKELRVIESGPHCANTEIIVKLSDGRELCLDPKENWVQRVVEKFLKRAEXS

SEQ ID NO: 14 - protein, 76 aa, Artificial sequence; identical to SEQ ID NO: 7
  Synthetic peptide

SEQ ID NO: 15 - protein, 74 aa, Artificial sequence; identical to SEQ ID NO: 8
  Synthetic peptide

SEQ ID NO: 16 - protein, 77 aa, Artificial sequence; identical to SEQ ID NO: 9
  Synthetic peptide

SEQ ID NO: 17 - protein, 68 aa, Artificial sequence
  Synthetic peptide
KPVSLSYRCPCRFFESHVARANVKHLKILNTPNCALQIVARLKNNNRQVCIDPKLKWIQEYLEKALNK

SEQ ID NO: 18 - protein, 72 aa, Artificial sequence
  Synthetic peptide
SAKELRCQCIKTYSKPFHPKFIKELRVIESGPHCANTEIIVKLSDGRELCLDPKENWVQRVVEKFLKRAENS

SEQ ID NO: 19 - protein, 73 aa, Artificial sequence
  Synthetic peptide
FPMFKRGRCLCIGPGVKAVKVADIEKASIMYPSNNCDKIEVIITLKENKGQRCLNPKSKQARLIIKKVERKNF

SEQ ID NO: 20 - protein, 73 aa, Artificial sequence
  Synthetic peptide
KSMQVPFSRCCFSFAEQEIPLRAILCYRNTSSICSNEGLIFKLKRGKEACALDTVGWVQRHRKMLRHCPSKRK

SEQ ID NO: 21 - protein, 69 aa, Artificial sequence
  Synthetic peptide
AQVGTNKELCCLVYTSWQIPQKFIVDYSETSPQCPKPGVILLTKRGRQICADPNKKWVQKYISDLKLNA

(19) United States
(12) Patent Application Publication
(10) Pub. No.: US 2004/0138422 A1
(43) Pub. Date: Jul. 15, 2004

(54) Synthetic chemokines labeled at selected positions

(76) Inventors: Stephane Demotz, Epalinges (CH); Corinne Moulon, Epalinges (CH); Christophe Reymond, Prilly (CH); Mario Roggero, Epalinges (CH); Andrew Strong, Lusanne (CH); Jean Vizzavona, Andilly (FR); Pascal Cousin, Savigny (CH)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/668,733

(22) Filed: Sep. 23, 2003

Related U.S. Application Data
(60) Provisional application No. 60/412,866, filed on Sep. 23, 2002.

(51) Int. Cl. C07K 14/52

(52) U.S. Cl. 530/351; 530/408

(57) ABSTRACT

Labeled synthetic chemokine molecules are provided which are characterized by improved biological activity and methods for their production and use in detection of chemokine receptors.

### RELATED APPLICATION

This application claims benefit of U.S. Provisional Patent Application Serial No. 60/412,866 filed Sep. 23, 2002, the entirety of which is incorporated by reference herein.

### BACKGROUND OF THE INVENTION

Chemokines constitute a protein family of over 40 members, which exhibit a wide variety of biological activities required in many normal physiological processes, but also implicating in pathological situations.

Over the last few years, the biology of chemokines has been the subject of intense research activities. In a short time span, many chemokines and their corresponding receptors have been identified. With the sequencing of the human genome and the advent of its computer-based analysis, several chemokines were indeed first identified through their gene, rather than isolation of the protein itself. It was soon realized that chemokines are involved in many physiological processes, such as cell migration, cell activation and angiogenesis, but also implicated in pathological manifestations, such as inflammation and AIDS. This situation prompted the pharmaceutical sector to attempt the isolation of low molecular weight compounds capable of interfering with the interaction between chemokines and their receptors. Obviously, the success of this undertaking relied on the availability of binding tests, which would be suitable for high throughput screenings.

Initially, binding assays were developed with the use of radiolabeled chemokines. Eventually, if successful, this approach is nowadays no longer a preferred option, due to the multiple problems associated with radioactive material handling. Alternative approaches have been contemplated, among them the use of haptens, such as biotin, for the tagging of chemokines. One problem encountered at that stage is the way of introducing the hapten without interfering with the biological activity of the chemokines, in particular the binding characteristics to their receptors. One approach relied on the modification of recombinant chemokine using chemically activated biotin. Even though straightforward, the major flaw of this method is the difficulty in controlling how much and where on the peptide the biotin moieties are introduced. As a result, the labeled chemokines have a markedly decreased biological activity as compared with the unmodified one.

Of interest to the present invention is Abdelaziz, Vita et al. (WO 0012554) which reports production of a labeled chemokine through chemical synthesis methods.

Also of interest is the report of labeling the N-terminus of a polypeptide in Synthesis And Characterization Of Fluorescent And Photoactivatable MIP-1 Alpha Ligands And Interactions With Chemokine Receptors CCR1 and CCR5, S. Zoffmann, G. Turcatti, J. Galzi, M. Dahl and A. Chollet, J Med Chem. 2001 Jan. 18;44(2):215-22; Synthesis Of Nonluminescent Lanthanide (III) Chelates Tethered To An Aminooxy Group And Their Applicability To Biomolecule Derivatization, J. Peuralahti et al., Bioconjugate Chem. 2002, 13:876-880; Synthesis And Evaluation Of Fluorescent Chemokines Labelled At The Amino Terminal, RE Offord et al., Methods Enzymol, 1997, 2287:348-369. Also of interest is the report of polypeptide synthesis in Design And Chemical Synthesis Of A Homogeneous Polymer-Modified Erythropoiesis Protein, G. G. Kochendoerfer et al., Science 2003, 299:884-887.

Alternatively, some authors have pursued an enzymatic approach of chemokine modification. Albeit ingenuous, this method is labor intensive and the tagged chemokine is obtained in low yield. In particular, it requires the difficult separation of the labeled from the unlabeled chemokine. In addition, if successful for some chemokines, this method is by far not applicable to any peptide ligand and is also limited by the availability and suitability of the substrates. Accordingly, there remains a desire in the art for improved methods for producing labeled chemokines having improved chemokine biological activity.

### SUMMARY OF THE INVENTION

The present invention is related to the discovery that chemically synthesized labeled polypeptides, such as chemokines, can be produced which are characterized by good label binding properties and surprising levels of biological activity including chemokine receptor binding activity. In one aspect of the present invention, labeled chemokines are produced by the method including the steps of forming a polypeptide chain by total chemical synthesis such as by means of a solid phase peptide synthesis (SPPS) strategy. According to one preferred aspect of the invention, the SPPS method is a 9-fluorenylmethyloxycarbonyl (Fmoc) solid phase peptide synthesis (SPPS) strategy utilizing 9-fluorenylmethyloxycarbonyl (Fmoc) as a protective moiety. Wellings and Atherton, Methods Enzymol. 289:44-67 (1997); Roggero et al., Eur. J. Immunol. 30:2679-85 (2000). Alternatively, synthesis can be performed in the liquid phase using Fmoc chemistry, or by solid or liquid phase using other chemistries such as CBz or Nsc which are known to those of skill in the art.

The method optionally comprises substituting an amino acid residue by a modified moiety at one or more selected positions on the polypeptide chain, where the modified moiety at the selected position(s) allows subsequent detection of the chemokine. Modified moieties can include label moieties such as fluorescent or biotin labels or can comprise activatable groups. In the method of the present invention, the modified moiety which is incorporated into the chemically synthesized peptide or polypeptide may be a modified moiety that is already linked to a label moiety, or may be a modified moiety that comprises an activatable group that is linkable, after activation, to a label moiety. Exemplary activatable groups according to this method include an ethan-1,2-diol, a beta-hydroxyamine or a beta-aminothiol group. The activatable group on the modified amino acid can then be used to covalently link the desired label to the modified amino acidin one preferred embodiment, the modified moiety is introduced in the form of a biotinylated Fmoc-protected lysine residue. In other preferred embodiments, the modified moiety is Dpr(Ser), Lys(Ser), or ornithine(Ser).

The method of the present invention may further comprise the step of activating the activatable group to an aldehyde or ketone group. Where the polypeptide comprises an amino-terminal residue containing an ethan-1,2-diol, a beta-hydroxyamine or beta-aminothiol group, the method may further comprise the steps of protecting the amino-terminal residue by adding a protecting group before the activation step, followed by removing the protecting group after the activation step. Exemplary protecting groups include an Fmoc, CBz or benzyl group. The method according to the invention may further comprise the step of linking the modified moiety to a hydrazine, aminooxy or beta-aminothiol group.

Preferably the amino acid positions at which the modified moieties are introduced are selected such that the labeled polypeptide produced according to the method of the present invention is strongly detectable. Preferably the labeled polypeptide also retains substantially the same biological activity as the unlabeled polypeptide.

In order to allow for subsequent detection of the peptide, activatable group on the modified moiety is reacted with one or more label moieties after formation of the polypeptide chain by the chemical synthetic method. According to one preferred aspect of the invention, the addition of the label moiety preserves active conformation of the peptide such that it retains the properties of receptor-binding, receptor activation and/or other biological activity.

Label moieties according to the invention can include radioactive labels, affinity labels (such as biotin, avidin or streptavidin, each of which is detectable through binding to its affinity partner), enzymatic labels (such as horseradish peroxidase, alkaline phosphatase), labels detectable through fluorescence (such as FITC or rhodamine or other fluorophore), lanthanide chelates, redox couples allowing electrochemical detection, or labels allowing magnetic detection such as paramagnetic atoms or paramagnetic groups. Labeled moieties detectable through fluorescence can include fluorescein or anyone of the family dyes AlexaFluor® (Molecular Probes Inc., Eugene, Oreg.), EVOblue® (Evotec OAI, Hambourg, Germany), Atto (Atto Bioscience, Rockville, USA), Cy® (Amersham Biosciences, Piscataway, N.J.), Europium chelates (Perkin Elmer, Boston, Mass., USA) and DY (Dyomics GmbH, Jena, Germany).

In one exemplary application of the method an Fmoc-protected lysine residue that is covalently linked to biotin is introduced at appropriate location(s) in the polypeptide chain. The lysine with covalently linked biotin is then reacted with fluorescently labeled avidin or streptavidin to allow detection. In another exemplary application, (dpr) serine containing an activatable group is introduced at appropriate location(s) in the polypeptide chain. The (dpr) serine is then activated into a beta-aminoaldehyde and reacted with fluorescein hydrazide or an hydrazide-AlexaFluor® dye to allow detection.

Thus, one aspect of the invention provides a method of producing a detectably labeled polypeptide (for example, a chemokine) retaining the receptor-binding specificity of unlabeled polypeptide comprising the step of introducing by chemical synthesis a modified moiety at one or more selected amino acid positions in said polypeptide. In one embodiment of the method, the entire labeled polypeptide is chemically synthesized. In an alternative embodiment of the method, the labeled polypeptide is formed by linking a recombinantly expressed peptide to a chemically synthesized peptide.

Because the location at which the peptides are labeled may be selected, the present method avoids the problems resulting from prior art methods which rely upon random labeling of the polypeptide backbone. Such random labeling can adversely affect the biological activity of the labeled molecule (including cytokine activity and cytokine receptor binding activity) while not necessarily presenting a label moiety, such as biotin, in a conformation available for binding with a label binding partner such as avidin. Accordingly, the methods of the present invention can utilize structural activity relationships known to those of skill in the art to design and select the placement of labeled peptide moieties to provide labeled, detectable, biologically active molecules. Such polypeptides containing label at selected positions, rather than random labeling, retain the receptor-binding specificity and receptor activation properties of the unlabeled polypeptide, and most preferably retain substantially the same biological activity of the unlabeled polypeptide when measured under the same conditions in the same assay (for example, a 2-fold difference or less).

The methods of the present invention also provide greater uniformity than random labeling methods. Preferably greater than 95%, 96%, 97%, 98% or 99% of the labels attached to the polypeptide are attached at the specific selected position(s).

Suitable assays for measuring specific chemokine receptor-binding activity include competition assays measuring the ability of the labeled chemokine to compete with unlabeled chemokine for binding to the desired receptor. Suitable assays for measuring chemokine receptor activation properties include assays measuring cell migration, chemotaxis, leukocyte degranulation, actin polymerization, cell shape modification, calcium flux induction, or enzyme release.

Labeled chemokine molecules may be used in a variety of therapeutic and diagnostic applications, including in chemokine receptor assays, including high throughput screening assays. Labeled chemokine can be used to screen for cells expressing the chemokine's receptor, for example, for identifying cell subtypes. Labeled chemokine can also be used in assays requiring detection of binding of labeled chemokine to its receptor and can be used to screen for inhibitors or enhancers of chemokine binding. Preferably such assays are whole cell assays conducted using cells expressing the appropriate chemokine receptor on its surface.

Another aspect of the present invention is a detectably labeled polypeptide, preferably a chemokine, that retains the biological activity of unlabeled polypeptide. A preferred embodiment is a labeled chemokine retaining substantially the same biological activity as unlabeled chemokine, wherein said labeled chemokine comprises a label moiety at a selected amino acid position that is not the N-terminus of the chemokine, and wherein said labeled chemokine is human CCL22 (native sequence is SEQ ID NO: 1), CCL2 (native sequence is SEQ ID NO: 14), CCL11 (native sequence is SEQ ID NO: 15), CCL19 (native sequence is SEQ ID NO: 16), CXCL12 (native sequence is SEQ ID NO: 17), CXCL11 (native sequence is SEQ ID NO: 19), CCL1 (native sequence is SEQ ID NO: 20), CCL18 (native sequence is SEQ ID N O: 21) or CXCL8 (native sequence is SEQ ID NO: 18). The label moiety is preferably within the C-terminal half of the chemokine. Such labeled polypeptides may be produced according to the methods of the invention described above.

In preferred embodiments, a labeled polypeptide according to the present invention is a labeled chemokine that is a synthetic analogue of human CCL22, CCL2, CCL11, CCL19, CXCL12, CXCL11, CCL1, CCL18 or CXCL8. In more preferred embodiments, the labeled chemokine is human CCL22 modified at position 66, human CCL2 modified at position 75, human CCL11 modified at position 73, human CCL19 modified at position 73, human CXCL12 modified at position 67, human CXCL8 modified at position 71, human CXCL11 modified at position 71, human CCL1 modified at position 71 or human CCL18 modified at position 66. In most preferred embodiments, the labeled chemokine is one set forth in any one of SEQ ID NOS: 6-13. Exemplary label moieties for the labeled polypeptides of the present invention include biotin, fluorophore, lanthanide chelate, redox couple, paramagnetic group, chromophore, or radioactive labels.

The invention also contemplates methods of detecting the presence of a receptor that binds to a labeled polypeptide of the invention, such methods comprising the steps of contacting said receptor with said labeled polypeptide and detecting the presence of said labeled polypeptide. In a preferred embodiment, the the presence of a chemokine receptor is detected using a labeled chemokine of the invention. The receptor thus detected may be a truncated soluble or chimeric variant or may be expressed on the surface of a cell. According to such methods, the detection of the presence of said receptor may be indicative of a cellular subtype.

The invention further contemplates a method of screening for a modulator of the binding of a polypeptide to a receptor comprising the steps of contacting said receptor with a labeled polypeptide according to the invention, in the presence and absence of a test compound, and detecting the relative amount of binding in the presence and absence of said test compound. Also contemplated is a method of screening for a modulator of receptor activation comprising the steps of contacting said receptor with a labeled polypeptide according to the invention, in the presence and absence of a test compound, and detecting the relative amount of receptor activation in the presence and absence of said test compound. The test compound may be an inhibitor or enhancer of receptor binding or receptor activation.

Binding of the labeled polypeptide, including a labeled chemokine, to a receptor may also be used in a method of sorting cells, comprising the steps of contacting a labeled polypeptide of the invention with said cells, and separating cells that bind said labeled polypeptide from cells that do not bind said labeled polypeptide. Such methods may use fluorescent-activated cell sorting techniques, magnetic beads, or avidin-coupled beads.

Binding of the labeled polypeptide, including a labeled chemokine, to a receptor may also be used in medical imaging methods comprising the steps of administering a labeled polypeptide according to the invention to a subject and detecting the location of said labeled polypeptide within the subject.

### BRIEF DESCRIPTION OF THE DRAWINGS

FIG. 1 shows results from a migration assay showing the effects of hCXCL12 and hCXCL12-AlexaFluor647 on migration of CXCR4-transfected cells.

FIG. 2 shows results from a fluorescence-activated cell sorting (FACS) analysis showing binding of hCXCL12-AlexaFluor647 labeled chemokine to CXCR4-transfected cells or untransfected B300.19 cells.

### DETAILED DESCRIPTION

The present invention provides improved methods for the production of labeled polypeptides, such as chemokines. A clear advantage of the methods for the production of biotinylated and other labeled polypeptides, e.g., chemokines, is the ability to control the number of added biotin or other label moieties and the position to which they are introduced along the polypeptide sequence. In contrast, chemokines of recombinant origin are usually modified with activated biotin which is indiscriminately reactive towards amino groups resulting in random labeling and loss of biological activity. Indeed, analysis of such a commercially available reagent found it to contain multiple molecular species and characterized by reduced biological activity. This situation is likely due to the modification of the N-terminal residue and lysine residues in the N-terminal region of the chemokine needed for binding to its receptor. Modifications of chemokines during step wise synthesis is not restricted to biotin, but can also be practiced with other haptens (e.g. DNP, digoxin) and other types of modifications (e.g. unnatural amino acids).

To the extent that biotinylated chemokines offer flexibility in terms of selection of secondary/detection reagents, they still require the use of a developing reagent. In this regard, a one step binding assay would be advantageous including for use such as for high throughput applications, such screening of large libraries of chemical compounds. According to one aspect of the invention, a preferred cell staining assay may be carried out with complexes between biotinylated chemokines and FITC-labeled avidin. Thus, staining can be achieved in a one step procedure.

Nevertheless, should the pharmacological behavior of chemokine and avidin complexes be different from that of monovalent chemokine the methods of the invention may be used to produce chemokines directly labeled with fluorescent dyes providing preferred properties in terms of ease of use and pharmacological features. Use of fluorochrome-labelled chemokines that contain a fluorescent label at selected position(s) allow a better quantitation of ligand bound to its receptor, compared to the use of biotinylated chemokines using a tetravalent secondary reagent (e.g., avidin or streptavidin).

Thus in another aspect of the invention a method is described for producing chemokines directly labelled with fluorescent dyes. An exemplary method uses a specific reaction between an aldehyde group on the chemokine and a hydrazide or aminooxy group on the dye. These groups react without modifying the other groups on the side-chains of a peptide or protein (T. P. King et al, Biochemistry, 1986, 25, 5774-5779). An advantage of this method is that modification occurs in solution following refolding of the chemokine into its native form, and therefore this essential folding step is not hindered by the bulky fluorescent group. Another advantage is that only small quantities of the fluorophore are needed, compared to the amount which would be necessary if labeling was made on the resin before cleavage. A further advantage of this method is that fluorophores or other groups such as lanthanide metal chelates, which are not stable to the acidic conditions of TFA cleavage may be used to label chemokines and retain biological activity. The directly labeled chemokines produced by this method have been found to stain cells expressing the appropriate chemokine receptor and had comparable activities to the native chemokines in a migration assay.

The Examples 1-3 set out below describe the production of five biotinylated CCL22 analogues and the use of one of them for the development of a whole cell binding assay. These results illustrate the advantage of the chemical synthesis approach for the production of tagged polypeptide ligands retaining their full biological activity. A chemokine binding assay on whole cells was developed using biotinylated synthetic CCL22 having the sequence (GPYGANMEDSVCCRDYVRYRLPLRVVKHFYWTSDSCPRPGVVLLTFRDKEI CADPRVPWVKMILNKLSQ) (SEQ ID NO: 1) as a model ligand. CCL22 analogues were produced by a chemical route, resulting in >97% homogenous and defined polypeptides. First, it was shown that the five biotinylated CCL22 analogues synthesized were captured by agarose-immobilized streptavidin, indicating that the biotin molecules introduced in positions G1, K27, K49, K61 and K66 of CCL22 (SEQ ID NOS: 2, 3, 4, 5 and 6, respectively) were accessible for binding. Then, it was established using a migration assay that these biotinylated chemokines were as biologically active as the unmodified CCL22 form. Subsequently, these biotinylated chemokines were evaluated in a FACS-based whole cell binding assay. Significantly, the CCL22 analogue with the biotin in position K66 was the best staining reagent of CCR4-positive cells, with robust staining detected. The CCL22 analogue with the biotin in position K27 was weakly detected on the cells, while the other CCL22 analogues (K49, K61 and G1) did not result in cell staining. Finally, binding characteristics of the CCL22 analogue with the biotin in position K66 were outlined. These results exemplified that biotinylated synthetic chemokines constitute promising ligands for the development of chemokine receptor binding assays on whole cells, provided the biotin moiety is introduced in a defined positions.

The Examples 4-7 describe the production and analysis of chemokines directly labelled with a fluorescent group or a lanthanide metal chelate. The chemokine is synthesised by standard methods of peptide chemistry, replacing one residue from the native sequence with an activatable residue, Dpr(Ser). The residue to be replaced is chosen so as not to affect the receptor binding or biological activity of the chemokine. Following synthesis on the solid phase and cleavage from the resin, the polypeptide is purified and refolded into its native conformation by methods which are known to those of skill in the art. The activatable Dpr(Ser) residue of the refolded chemokine is then activated by oxidation with sodium periodate to the oxalyl group and purified. The aldehyde moiety of this oxalyl group is able to react specifically with complementary groups such as aminooxy, hydrazine or beta aminothiol. The examples 4 and 6 describe reaction of chemokines activated to an oxalyl group with a fluorescent hydrazine derivative or with a lanthanide metal chelate bearing an aminooxy group and then purified to produce chemokines labeled at a specific position with a detectable group. Purification may be done by RP-HPLC and ion-exchange chromatography by methods which are known to those of skill in the art. However to purify chemokines labeled with the fluorescent dye Alexa Fluor®647 purification by cation-exchange chromatography is the preferred method. The example 4 describes how human CXCL12 labeled with the fluorescent group Alexa Fluor®647 was shown to have similar biological activity in a migration assay to that of the native chemokine and stained cells expressing the appropriate receptor, human CXCR4.

Polypeptides may also be labeled with detectable groups which are not commercially available in the hydrazide, aminooxy or beta-aminothiol form. The example 5 describes the modification of a fluorescent dye, available as the free acid or active ester, to have an aminooxy group. The dye is reacted with the N-alpha moiety of the resin-bound amino acid Dpr(Boc-aminooxyacetyl) to form a peptide bond between the dye and the aminooxy reagent. Treatment with trifluoroacetic acid deprotects the aminooxy group and cleaves the aminooxy-dye reagent from the resin.

The example 7 describes how a polypeptide may be specifically labeled at one position if the N-terminal residue is also sensitive to the activation conditions. For example Ser, Thr or reduced Cys at the N-terminal of a polypeptide labeled elsewhere in the sequence with Dpr(Ser) would also be oxidised to an oxalyl group by periodate treatment during the activation step. To prevent this the N-terminal residue must be protected during the activation step, and the protecting group subsequently removed. The example 7 describes how the N-terminal residue of the human chemokine IL-8 (CXCL8) may be protected by the Fmoc group before the activation of the Dpr(Ser) with periodate reagent. After quenching of the excess periodate at the end of the reaction, the Fmoc group is removed in situ by treatment with 36% piperidine for 20 min, followed by purification. The mono-oxalyl chemokine was then labeled with the fluorescent dye Alexa Fluor®647 hydrazide.

### EXAMPLE 1

### Synthesis of Biotinylated Human CCL22

According to this example, synthetic human CCL22 was assembled using solid phase Fmoc chemistry as described in Roggero et al., Eur. J. Immunol. 30:2679-85 (2000), Wellings and Atherton, Methods Enzymol. 289:44-67 (1997). Biotin was introduced during synthesis under the form of biotinylated Fmoc-protected lysine residues (in positions K27, K49, K61 or K66, SEQ ID NOS: 3, 4, 5, and 6, respectively)) or by modification of the N-terminal residue (G1, SEQ ID NO: 2) with biotin N-succinimidyl ester (Fluka, Buchs, Switzerland). Following trifluoroacetic cleavage from the resin, the crude polypeptide was fractionated by RP-HPLC and lyophilised as described in Roggero et al., Eur. J. Immunol. 30:2679-85 (2000).

The crude polypeptide was solubilized in 3 M guanidium. HCl, 50 mM Na2HPO4 and 5 mM Tris, pH 8.0 in the presence of a 100-fold molar excess of cysteine over peptide. After 1 day of incubation at 37° C., oxidation was stopped by the addition of TFA to a final concentration of 0.2% and the chemokines were isolated by RP-HPLC on a Vydac C18 column with a linear gradient of acetonitrile in water, 0.1% trifluoroacetic acid. Purity and identity of the chemokines were finally assessed by RP-HPLC and mass spectrometry. Analytical RP-HPLC showed that the different chemokines eluted as a single, symmetrical peak, indicating a purity >97%. Mass spectrometry established that the synthetic material contained one molecular species of the expected molecular mass.

### EXAMPLE 2

### Activity of Biotin-Labeled CCL22

According to this example, samples of the biotin-labeled CCL22 molecules produced according to Example 1 were subjected to various assays of binding ability and biological activity.

Streptavidin Capture Assay

In order to assess whether the biotin moiety introduced in the different positions a long the sequence of CCL22 was accessible for binding, samples of the five biotinylated CCL22 analogues, as well as the unmodified form, were incubated in the presence or the absence of streptavidin-agarose. Specifically, samples of the chemokines (25 μg in 175 μl PBS) were subjected to a streptavidin-agarose capture assay in which they were incubated for 1 h at room temperature under continuous agitation in the presence or the absence of 50 μl of streptavidin-agarose (Sigma, St. Louis, Mo.). Then, the resin was discarded and the samples analysed by RP-HPLC.

Subsequently, the amount of chemokine left in the samples was evaluated by RP-HPLC. For the five biotinylated CCL22 species tested, incubation with streptavidin-agarose resulted in the complete disappearance of the chemokine. When no pre-incubation with streptavidin-agarose was applied, the five chemokine species eluted as single peaks. By contrast, the unmodified CCL22 form was not bound by streptavidin-agarose. Together, these results indicated that for the five biotinylated analogues, the biotin moiety was accessible for binding by streptavidin.

Migration Assay

The biological activities of the five biotinylated CCL22 analogues were then evaluated for their capacity to bind to and to induce migration of CCR4-transfected cells. CCR4-transfected B lymphoma cells were obtained as described in Loetscher et al. (J. Biol. Chem. 276:2986, 2001) and cultured in RPMI 1640 (Gibco, Paisley, UK) supplemented with 10% FCS and 1.5 μg/ml puromycin (Sigma). Migration induced by the CCL22 analogues was determined using 5 μm polycarbonate Transwell inserts (Costar, Corning, N.Y.). Briefly, 3×10⁵ cells in 1001 μl RPMI 1640 and 0.5% BSA were dispensed into the upper chambers of the Transwell. Chemokine in the same medium was added to the lower chamber in a final volume of 600 μl. After 3 h incubation at 37° C., cell migration was determined using an LDH-based colorimetric assay (Promega, Madison, Wis., CytoTox 96 non-radioactive cytotoxicity assay) on the cells recovered in the lower chamber. Results are given as mean O.D. at 490 nm. Each experiment was repeated at least three times.

Control experiments showed that CCL22 did not induce migration of the untransfected cells. Similarly to the bell-shaped dose response observed with unmodified CCL22, the five biotinylated CCL22 analogues promoted optimal migration of CCR4-transfected cells at a dose of 1 to 10 ng/ml and minimal migration still detectable at 0.1 ng/ml. These results showed that the biotin molecule introduced in the five positions evaluated did not impair the biological activity of the chemokine as evaluated by the ability to induce migration of CCR4 positive cells and suggest that the characteristics of binding between CCL22 and CCR4 were minimally impacted by the biotin molecules singly introduced in these five different positions.

Cell Staining

CCR4-transfected or untransfected B300.19 mouse lymphoma cells in staining buffer (PBS with 5% AB serum) were incubated for 30 min at 4° C. in the presence or the absence of 1 μg/ml biotinylated anti-human CCR4 mAb (1G1.1, BD Biosciences, San Jose, Calif.). The cells were then washed with staining buffer and incubated with 100 ng/ml PE-conjugated streptavidin (Biosource International, Nivelles, Belgium) for 30 min at 4° C. After 2 washing with staining buffer, the cells were fixed in PBS with 1% FCS 1% and 1% PFA. Cell analysis was performed on a FACSCalibur cytofluorometer (BD Biosciences).

For staining using biotinylated chemokine, 3×10⁵ CCR4-transfected or untransfected B300.19 cells were incubated with biotinylated h-MDC at the indicated concentrations for 1 hour at 4° C., followed by incubation with a FITC-conjugated avidin (BD Biosciences) at the indicated concentrations. After 2 repetitive washings using staining buffer, cells were fixed in PBS with 1% FCS 1% and 1% PFA before FACS analysis.

Staining of the CCR4-transfected cells using a biotinylated anti-CCR4 mAb and FITC-labeled avidin as a secondary reagent revealed the presence of CCR4 on these cells. In contrast, no CCR4 expression was detected on the untransfected cells. The presence of bound chemokine on the CCR4-positive cells was revealed by the addition of FITC-labeled avidin. A staining as intense as the one obtained with biotinylated anti-CCR4 mAb was observed following incubation of CCR4-transfected cells with biotinylated CCL22(K66). By contrast, no staining was detected on the untransfected cells. These results indicated that the staining was due to the binding of biotinylated CCL22 to CCR4.

The four other biotinylated analogues were also evaluated for their ability to work as FACS staining reagents. Surprisingly, only the CCL22(K66) analogue resulted in robust staining of CCR4-positive cells. By contrast, the biotinylated CCL22(K27) analogue was faintly detected (although not as efficiently as K66), and the biotinylated CCL22(G1), CCL22(K49) and CCL22(K61) analogues did not result in staining of CCR4-transfected cells (See Table 1A).

Since the biotinylated CCL22(K66) analogue was found the best suitable staining reagent, its binding characteristics were further delineated. First, its binding was shown to be impeded by pre-incubation of CCR4-transfected cells with increasing concentrations of unmodified CCL22. These results established the saturable nature of the CCL22 receptor expressed on these cells (Table 1B). The sensitivity of the staining was then evaluated. Weak staining was detected with concentrations of biotinylated CCL22(K66) analogue as low as 10 ng/ml. Staining intensity reached saturation at 300 ng/ml of biotinylated CCL22(K66) analogue (Table 1C). The former concentration corresponded to a concentration eliciting maximal migration of CCR4-transfected cells, indicating that cell staining represented a relevant correlate for migration. Finally, it was evaluated whether the staining procedure could be made easier by performing complexes between the biotinylated CCL22(K66) analogue and FITC-avidin prior to addition to cells. A simplified staining procedure would result in increasing throughput of samples in this staining assay. It was found that when made at a one to one molar ratio, biotinylated CCL22(K66) analogue and FITC-avidin complexes resulted in a positive whole cell staining (Table ID). In contrast, when biotinylated CCL22(K66) analogue and FITC-avidin were pre-incubated at a molar ratio lower or higher than one, no staining of CCR4-transfected cells was obtained.

Discussion

The data above demonstrate the development of a non-radioactive chemokine binding assay on whole cells. Using a FACS-based detection system, binding between CCR4 and CCL22 using a biotinylated form of CCL22 was visualized with a fluorescence intensity stronger than the one obtained with anti-CCR4 antibodies. This difference could be explained by the high biotin content of the mAb (5-10 biotin/mAb) as compared with the synthetic chemokine (1 biotin/chemokine). Surprisingly, only one biotinylated CCL22 analogue, out of the five tested, constituted a suitable reagent for CCR4 staining on whole cells. Initially, it was visually evaluated using the RasMol molecular graphics visualisation program (www.rasmol.org) that where introduced, the biotin moieties was pointing towards the solvent and thereby likely accessible for binding by a capture protein, such as streptavidin.

This was confirmed by the fact that streptavidin-agarose was found to efficiently bind the five biotinylated CCL22 analogues. In addition, since these five CCL22 analogues exhibited a migration-inducing capacity similar to the one of the unmodified CCL22 molecule, one may assume that these different CCL22 species did indeed bind to CCR4. Work from other investigators identified patches of residues on the chemokine, which are needed for binding to the receptor. Hemmerich et al., Biochemistry, Oct 5; 38(40): 13013-25 (1999); Jamagin et al., Biochemistry 38:16167-77 (1999). Based on the structural similarities between chemokine species, it is estimated that the residues used for biotinylation are not the ones directly in contact with CCR4. This is indeed likely, since the biotinylated CCL22 analogues were found to exhibit full biological activity. Rather, the failure to detect these chemokines in a FACS-based assay was more likely due to an accessibility limitation for the detecting reagent (FITC-avidin). A reason explaining why four of the biotinylated CCL22 analogues, while bound to their receptor, cannot be visualized by FITC-labeled avidin, could be that the biotin moiety became no longer accessible for binding by avidin. Similar results were obtained in a microscopy-based application (FMAT, Applied Biosystems). This is indirectly supported by the observation that other secondary reagents than FITC-labeled avidin, such as FITC-streptavidin, PE-labeled streptavidin or Alexa Fluor 488-labeled anti-biotin mAb, failed to detect biotinylated CCL22(K66) bound onto CCR4 positive cells.

TABLE 1A

| | MFI | MFI |
|---|---|---|
| CCL22 Analogues | Untransfected Cells | CCR4-Transfected Cells |
| G1 (N-terminal) | 5.4 | 5.9 |
| SEQ ID NO: 2 | | |
| K27, SEQ ID NO: 3 | 5.1 | 8.7 |
| K49,, SEQ ID NO: 4 | 5.3 | 6.1 |
| K61, SEQ ID NO: 5 | 5.2 | 6.6 |
| K66, SEQ ID NO: 6 | 5.2 | 18.6 |
| Unbiotinylated CCL22 | 5.0 | 5.4 |

TABLE 1B

| Unlabeled CCL22 | MFI |
|---|---|
| (μg/ml) | CCR-4-Transfected Cells |
| 10,000 | 5.3 |
| 3,000 | 5.7 |
| 1,000 | 6.0 |
| 300 | 6.9 |
| 100 | 7.2 |
| 30 | 7.8 |
| 0 | 10.4 |
| Unbiotinylated CCL22 | 4.3 |

TABLE 1C

| CCL22 (K66) | MFI |
|---|---|
| (μg/ml) | CCR4-Transfected Cells |
| 300 | 11.0 |
| 100 | 9.9 |
| 30 | 7.5 |
| 10 | 5.7 |
| 3 | 5.2 |
| 1 | 5.0 |
| 0.3 | 5.0 |
| 0 | 4.7 |

TABLE 1D

| | | MFI | MFI |
|---|---|---|---|
| | | Untransfected | CCR4-Transfected |
| Staining Reagents | Staining | Cells | Cells |
| FITC-avidin | 2 steps | 2.5 | 3.0 |
| Biotinylated | 2 steps | 2.8 | 6.4 |
| CCL22(K66) + | | | |
| FITC-avidin | | | |
| FITC-avidin | Complexes | 2.5 | 3.1 |
| Biotinylated | Complexes | 2.6 | 8.3 |
| CCL22(K66) + | | | |
| FITC-avidin | | | |

### EXAMPLE 3

### Synthesis of Other Biotin-Labeled Chemokines

According to this example, biotinylated human chemokine species other than biotinylated CCL22/MDC, were produced, namely biotinylated CCL2/MCP-1 (the biotin moiety was introduced in position 75) (SEQ ID NO: 7), biotinylated CCL11/Eotaxin (the biotin moiety was introduced in position 73) (SEQ ID NO: 8) and CCL19/MIP-3beta/ELC (the biotin moiety was introduced in position 73) (SEQ ID NO: 9) were produced according to the methods of Example 1. Subsequently, a whole cell binding assay was developed using cell lines transfected with the corresponding chemokine receptors, namely CCR2 for CCL2, CCR3 for CCL11 and CCR7 for CCL19. While it was found that the binding conditions (e.g. temperature, period of incubation, presence or absence of inhibitors of the respiratory chain, preformed complexes between the biotinylated chemokine and the fluorescent secondary reagent or sequential addition of the biotinylated chemokine and of the fluorescent secondary reagent) are different for each assay those of ordinary skill in the art would be able to produce the biotinylated chemokines and utilize them in assay methods of the invention with little experimentation.

Binding of other biotinylated chemokines, such as CCL1, CCL5 and CCL11, was found to be considerably weaker, if not absent, even though receptor expression was high and the concentration of chemokine needed to elicit migration was low as in the cases in which binding was detected (CCL22 and CCL19). This failure could be explained by the fact that the biotin was not positioned adequately on these analogues or alternatively, the binding characteristics of these chemokines could be different from the ones successfully detected, such as CCL22 and CCL19. Experiments indicated that by raising temperature to 37° C. and using preformed CCL2-Biotin/Avidin-AlexalFluor488 complexes, CCL2 binding to CCR2 can be detected. This could be of particular importance for the detection of chemokines exhibiting biological activity at high concentration (μg/ml range), such as CXC chemokines, as compared with other ones, such as CCL22, active in the ng/ml range.

### EXAMPLE 4

### Synthesis and Activity of Fluorescent Human CXCL12/SDF-1-AlexaFluor®647

According to this example, synthetic human CXCL12 (SDF-1) labelled with Alexa Fluor®647 at position Asn67 was prepared. The peptide sequence, SEQ ID NO: 10, was assembled in the same manner as for CCL22 in Example 1 except that Asn67 was replaced by a (N^{β}-serine)diaminopropionic acid residue [Dpr(Ser)], introduced during the synthesis as Fmoc-Dpr(Boc-Ser(tBu))-OH (Novabiochem, Laufelfingen, Switzerland). Following synthesis, the peptide was cleaved from the resin, fractionated by RP-HPLC and refolded with formation of the disulphide bridges as described in Example 1, except that the concentration of guanidinium.HCl used to solubilise the crude polypeptide was 2M. After purification and lyophilisation, the chemokine's Dpr(Ser) group was transformed to an aldehyde group by reaction with sodium periodate. Thus the chemokine polypeptide (2-3 mg/ml in 50 mM imidazole buffer, pH 7.0) was oxidized for 5 minutes with a 5-fold molar excess of NaIO₄. Oxidation was terminated by addition of 10% ethylene glycol and 10% acetic acid in water and the chemokine was purified by RP-HPLC and lyophilised. The chemokine-aldehyde was solubilized at 2-5 mg/ml in 100 mM sodium acetate, pH 5, and reacted with 3 molar excess of Alexa Fluor® 647 hydrazide (Molecular Probes, Eugene, Oreg.). After overnight incubation at room temperature, the fluorescent chemokine was fractionated by cation exchange chromatography (MonoS column run on an AKTA purifier (Pharmacia Biosciences, Uppsala, Sweden), by forming a 0 to 1 M NaCl gradient in 20 mM sodium phosphate, pH 7.0. Homogeneity and identity of the chemokine species were assessed by analytical RP-HPLC and mass spectrometry (MALDI-TOF, Voyager Elite, Applied Biosystems, Foster City, Calif.).

Biological Activity:

SDF-1-alpha (CXCL12)-AlexaFluor®647 was evaluated for its capacity to bind to and induce migration of CXCR4-transfected cells. Control untransfected B300.19 cells and CXCR4-transfected B lymphoma cells were obtained as described in Loetscher et al. (J. Biol. Chem. 276:2986, 2001) and cultured in RPMI 1640 medium supplemented with glutamine, nonessential amino acids, sodium pyruvate, kanamycine, 5×10⁻⁵ M 2-mercaptoethanol (all from InVitrogen, Basel, Switzerland), 10% fetal calf serum (FCS), and 1.5 μg/mL puromycin (Sigma).

Migation Assay:

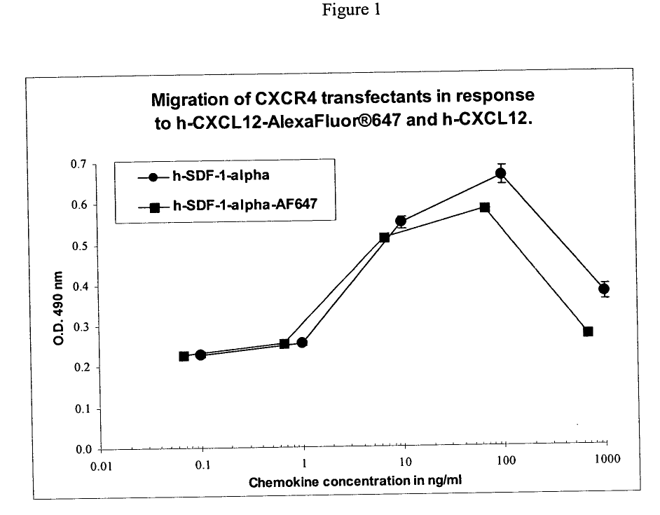

Migration induced by the CXCL12-AlexaFluor®647 was determined using 5 μm polycarbonate Transwell inserts (Costar, Corning, N.Y.). Briefly, 3×10⁵ cells in 100 μl RPMI 1640 and 0.5% bovine serum albumin were dispensed into the upper chambers of the migration wells. Chemokine was added at different concentrations in the same medium to the lower chambers in a final volume of 600 μl. For comparison of the biological activities, native non-labeled CXCL12 was also tested in the same assay. After 3 h incubation at 37° C., cell migration was determined using a lactate dehydrogenase assay-based colorimetric assay (CytoTox 96 nonradioactive cytotoxicity assay, Promega, Madison, Wis.) on the cells recovered in the lower chambers. Results are given as means of duplicate determinations of the optical density (OD) at 490 nm. In this assay, a higher OD indicates greater migration. The obtained results (shown in FIG. 1) indicate that the biological activity of h-CXCL12-AlexaFluor®647 remains intact in spite of the fluorochrome added on the chemokine. Both native chemokine and AlexaFluor®647-labeled CXCL12 were able to induce the migration of the h-CXCR4-transfected murine pre-B cell line 300-19 with a maximal migration occurring close to the 100 ng/ml dose.

Cell Staining:

For staining using AlexaFluor®)647-labeled chemokine, 3×10⁵ CXCR4-transfected or untransfected B300.19 cells were incubated with AlexaFluor®647-labelled CXCL12 at the indicated concentrations for 90 minutes at 4° C. in PBS containing 3% FCS. After 2 repetitive washings with PBS, cells were fixed in PBS containing 1% FCS and 1% paraformaldehyde before fluorescence-activated cell sorting (FACS) analysis. The fluorescence intensity of h-CXCL12-AlexaFluor®647 bound to h-CXCR4-transfected cells was measured in FL4 using a BD FACSCalibur™ Flow Cytometry system.

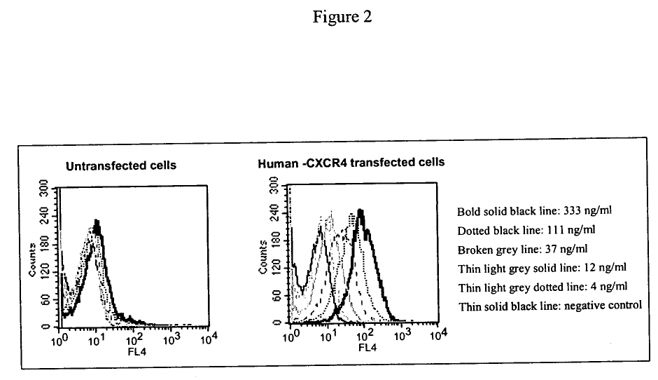

A dose titration was performed (shown in FIG. 2) and indicated that binding could be detected at a dose as low as 4 ng/ml and that fluorescence intensity increased together with increasing doses of the labelled chemokine (up to 333 ng/ml). Binding specificity was shown by the absence of staining of the parental non-transfected cells (B300.19 cell line).

### EXAMPLE 5

### Synthesis of Fluorescent Human CCL11/Eotaxin-EVOblue30

According to this example, a fluorescent dye which is not commercially available as a hydrazide, EVOblue30, is modifed with a short peptide bearing an aminooxy group, which may then react with a chemokine aldehyde of human CCL11 to produce the chemokine-dye conjugate linked by an oxime bond.

Fmoc-Dpr(Boc-Aoa)-OH(N-alpha-Fmoc-N-beta-(N-t.Boc-aminooxyacetyl)-L-diaminopropionic acid, from Novabiochem, Laufelfingen, Switzerland), was coupled to a Novasyn TGR resin (Novabiochem) with HOBt and DIPC in DMF. The Fmoc group was removed with 20% piperidine-DMF and EVOblue30 NHS ester (Evotec, Hamburg, Germany) was coupled to the free N-alpha in DMF. The resin-bound dye-peptide was then cleaved using TFA-water (19-5) and purified by RP-HPLC as described in Example 1 to afford the EVO-aminooxy reagent.

Human CCL11, SEQ ID NO: 11, bearing a Dpr(Ser) in place of Lys73 was prepared and folded as described for human CCL22 in example 1, except that the concentration of guanidinium.HCl used to solubilise the crude polypeptide was 0.6M. The chemokine polypeptide (2-3 mg/ml in 50 mM imidazole buffer, pH 7.0) was oxidized for 5 minutes with a 5-fold molar excess of NaIO₄ in the presence of a 500-fold molar excess of methionine with respect to the chemokine. Oxidation was terminated by addition of 10% ethylene glycol and 10% acetic acid in water and the chemokine was purified by RP-HPLC and lyophilised. The chemokine aldehyde was then labeled using the EVO-aminooxy reagent in place of Alexa Fluor® 647 hydrazide, using the protocol described for human CXCL12 in example 4. Purification was by RP-HPLC as described in Example 1 or by ion-exchange chromatography as described in Example 4.

### EXAMPLE 6

### Synthesis of Human CCL22/MDC-Europium Chelate

Human CCL22 bearing a Dpr(Ser) in place of Lys66 (SEQ ID NO:12) was synthesized and folded as described for human CCL22 in example 1. The chemokine was then oxidized with periodate reagent under conditions described in example 5. The oxidized human CCL22 was specifically labeled as follows: oxidized human CCL22 containing the aldehyde function at the above-mentioned-position is mixed with a 1.6 fold excess of Europium Chelate (J. Peuralahti et al., Bioconjugate Chem. 2002, 13:876-880) containing an amino-oxy group (Perkin Elmer, Finland) in a solution of ammonium acetate buffer (ammonium acetate 10 mM, 30% CH₃CN, pH 5) at room temperature. The labeling reaction is stirred, protected from light and incubated at room temperature. HPLC monitoring shows complete conversion of CCL22 into CCL22-Europium within 48 h. The solution is then directly freezed in liquid nitrogen and lyophilized with no further purification steps.

### EXAMPLE 7

### Synthesis of Fluorescent Human CXCL8/IL8

Human CXCL8 bearing a Dpr(Ser) in-place of Asn71, SEQ.ID NO: 13, was made and folded as described for human CCL22 in example 1, except that no deprotection of the N-terminal Fmoc-Ser(tBu) residue was made before cleavage of the peptide with TFA. Temporary protection prevents oxidation of the N-terminal serine into an aldehyde during subsequent periodate treatment.

Synthesis of the Precursor CXCL8-(Dpr-Aldehyde):

After production of the folded Fmoc-CXCL8-(Dpr-Ser) compound, the latter was first dissolved in an imidazole buffer (imidazole 50 mM, 10% CH₃CN, pH 7.0), then oxidized with ten-fold excess of periodate (NaIO₄) reagent (see example 5 for details). 5 min. later, the reaction was quenched with a 10% ethyleneglycol solution, left 2 min. with no stirring in order to add piperidine reagent to a final concentration of 36%. After 20 min. at room temperature under stirring, the solution was cooled in an ice-bath (0° C.) before the acidification with TFA (10% TFA solution followed by neat TFA). This solution is then HPLC purified on a C18 column as described in Example 1, directly freezed after elution from HPLC in liquid nitrogen and lyophilized.

Synthesis of CXCL8 Labeled with AlexaFluor647 Dye:

The precursor CXCL8-(Dpr-aldehyde) was mixed in acetate buffer (50% DMSO/sodium acetate 10 mM, pH 5.5) at room temperature. Then AlexaFluor647 hydrazide was added to the solution containing the precursor. The reaction was protected from light with aluminum foil and left 48 h at room temperature before HPLC monitoring. In this case, the labeling of human CXCL8 was successful, but was however incomplete as confirmed by HPLC and mass spectrum analyses. The CXCL8-Alexa Fluor 647 was further purified by RP-HPLC.

Numerous modifications and variations of the above-described invention are expected to occur to those of skill in the art. Accordingly, only such limitations as appear in the appended claims should be appended thereon.

### REFERENCES

1. Andjelkovic, A. V., Kerkovich, D., Shanley, J., Pulliam, L., Pachter, J. S. 1999. Expression of binding sites for beta chemokines on human astrocytes. Glia 28:225-35.

2. Andjelkovic, A. V., Spencer, D. D., Pachter, J. S. 1999. Visualization of chemokine binding sites on human brain microvessels. J. Cell Biol. 145:403-12.

3. Fernandez, E. J., Lolis, E. 2002. Structure, function, and inhibition of chemokines. Annu. Rev. Pharmacol. Toxicol. 42:469-499.

4. Hayes, I. M., Jordan, N.J., Towers, S., Smith, G., Paterson, J. R., Earnshaw, J. J., Roach, A. G., Westwick, J., Williams, R. J. 1998. Human vascular smooth muscle cells express receptors for CC chemokines. Arterioscler. Thromb. Vasc. Biol. 18:397-403.

5. Hemmerich, S., Paavola, C., Bloom, A., Bhakta, S., Freedman, R., Grunberger, D., Krstenansky, J., Lee, S., McCarley, D., Mulkins, M., Wong, B., Pease, J., Mizoue, L., Mirzadegan, T., Polsky, I., Thompson, K., Handel, T. M., Jarnagin, K. 1999.

6. Jarnagin, K., Grunberger, D., Mulkins, M., Wong, B., Hemmerich, S., Paavola, C., Bloom, A., Bhakta, S., Diehl, F., Freedman, R., McCarley, D., Polsky, I., Ping-Tsou, A., Kosaka, A., Handel, T. M. 1999. Identification of surface residues of the monocyte chemotactic protein 1 that affect signaling through the receptor CCR2. Biochemistry 38:16167-77.

7. Loetscher, P., Pellegrino, A., Gong, J. H., Mattioli, I., Loetscher, M., Bardi, G., Baggiolini, M., Clark-Lewis, I. 2001. The ligands of CXC chemokine receptor 3, I-TAC, Mig, and IP10, are natural antagonists for CCR3. J. Biol. Chem. 276:2986-91.

8. Roggero, M. A., Meraldi, V., Lopez, J. A., Eberl, G., Romero, J. C., Matile, H., Betschart, B., Corradin, G., Renggli, J. 2000. The synthetic, oxidized C-terminal fragment of the Plasmodium berghei circumsporozoite protein elicits a high protective response. Eur. J. Immunol. 30:2679-85.

9. Sallusto, F., Mackay, C. R., Lanzavecchia, A. 2000. The Role of Chemokine Receptors in Primary, Effector, and Memory Immune Responses. Annu. Rev. Immunol. 18:593-620.

10. Rossi, D., Zlotnik, A. 2000. The Biology of Chemokines and their Receptors. Annu. Rev. Immunol. 18:217-242.

11. Yoshimura, T., Leonard, E. J. 1990. Identification of high affinity receptors for human monocyte chemoattractant protein-1 on human monocytes. J. Immunol. 145:292-7.

12. Zhu, L., Bisgaier, C. L., Aviram, M., Newton, R. S. 1999. 9-cis retinoic acid induces monocyte chemoattractant protein-1 secretion in human monocytic THP-1 cells. Arterioscler. Thromb. Vasc. Biol. 19:2105-11.

13. Wellings, D. A., Atherton, E. 1997 Standard Fmoc Protocols. Methods Enzymol. 289:44-67.

| SEQUENCES | |
|---|---|
| CCL22: GPYGANMEDSVCCRDYVRYRLPLRVVKHFYWTSDSCPRPGVVLLTFRDKEIC | (SEQ ID NO.1) |
| ADPRVPWVKMTLNKLSQ | |
| CCL2: QPDAINAPVTCCYNFTNRKISVQRLASYRRITSSKCPKEAVIFKTIVAKEICADP | (SEQ ID NO.14) |
| KQKWVQDSMDHLDKQTQTPKT | |
| CCL11: ASVPTTCCFNLANRKIPLQRLESYRRITSGKCPQKAVIFKTKLAKDICADPK | (SEQ ID NO.15) |
| KKWVQDSMIKYLDQKSPTPKP | |
| CCL19: GTNDAEDCCLSVTQKPIPGYIVRNFHYLLIKDGCRVPAVVFTTLRGRQLCAPP | (SEQ ID NO.16) |
| DQPWVERIIQRLQRTSAKMKRSS | |
| CXCL12: KPVSLSYRCPCRFFESHVARANVKHLKILNTPNCALQIVARLKNNNRQVCIDP | (SEQ ID NO.17) |
| KIKWIQEYLEKALNK | |
| CXCL8: SAKELRCQCIKTYSKPFHPKFIKELRVIESGPHCANTEIIVKLSDGRELCLDPKE | (SEQ ID NO.18) |
| NWVQRVVEKFLKRAENS | |
| CXCL11: FPMFKRGRCLCIGPGVKAVKVADIEKASIMYPSNNCDKIEVIITLKENKGQRC | (SEQ ID NO.19) |
| LNPKSKQARLIIKKVERKNF | |
| CCL1: KSMQVPFSRCCFSFAEQEIPLRAILCYRNTSSICSNEGLIFKLKRGKEACALDTV | (SEQ ID NO.20) |
| GWVQRHRKMLRHCPSKRK | |
| CCL18: AQVGTNKELCCLVYTSWQIPQKFIVDYSETSPQCPKPGVILLTKRGRQICADP | (SEQ ID NO.21) |
| NKKWVQKYISDLKLNA | |

What is claimed:

1. A method of producing a detectably labeled polypeptide retaining the receptor-binding specificity of unlabeled polypeptide comprising the step of introducing by chemical synthesis a modified moiety at one or more selected amino acid positions in said polypeptide.

2. The method of claim 1 wherein the polypeptide is a chemokine.

3. The method of claim 1 wherein the entire labeled polypeptide is chemically synthesized.

4. The method of claim 1 wherein the labeled polypeptide is formed by linking a recombinantly expressed peptide to a chemically synthesized peptide.

5. The method of claim 1 wherein the modified moiety is a biotinylated amino acid.

6. The method of claim 1 wherein the modified moiety comprises an activatable group.

7. The method of claim 6 wherein the activatable group is an ethan-1,2-diol, a beta-hydroxyamine or a beta-aminothiol.

8. The method of claim 6 wherein the modified moiety is Dpr(Ser), Lys(Ser), or omithine(Ser).

9. The method of claim 6 further comprising the step of activating the activatable group to an aldehyde or ketone group.

10. The method of claim 9 wherein the polypeptide comprises an amino-terminal residue containing an ethan-1,2-diol, a beta-hydroxyamine or beta-aminothiol group, said method further comprising the steps of protecting the amino-terminal residue by adding a protecting group before the activation step, followed by removing the protecting group after the activation step.

11. The method of claim 10 wherein the protecting group is an Fmoc, CBz or benzyl group.

12. The method of claim 6 further comprising the step of linking the modified moiety to a hydrazine, aminooxy or beta-aminothiol group.

13. The method of claim 1 wherein the modified moiety is linked to a label moiety.

14. The method of claim 13 wherein the label moiety is biotin, avidin, streptavidin, a fluorophore, a lanthanide chelate, a redox couple, a paramagnetic group, a chromophore, or a radioactive label.

15. The method of claim 1 wherein the modified moiety is introduced in the form of a biotinylated Fmoc-protected lysine residue.

16. The method of claim 1 wherein the labeled polypeptide retains substantially the same biological activity as the unlabeled polypeptide.

17. A labeled chemokine produced according to the method of any one of claims 1-16.

18. A labeled chemokine produced according to the method of claim 1 which is a labeled synthetic analogue of human CCL22, CCL2, CCL11, CCL19. CXCL12, CXCL1, CCL1, CCL18 or CXCL8.

19. The labeled chemokine of claim 18 which is selected from the group consisting of human CCL22 modified at position 66, human CCL2 modified at position 75, human CCL11 modified at position 73, human CCL19 modified at position 73, human CXCL12 modified at position 67, human CXCL8 modified at position 71, human CXCL11 modified at position 71, human CCL1 modified at position 71 and human CCL18 modified at position 66.

20. The labeled chemokine of claim 18 selected from the group consisting of SEQ ID NOS: 6-13.

21. A labeled chemokine retaining substantially the same biological activity as unlabeled chemokine, wherein said labeled chemokine comprises a label moiety at a selected amino acid position that is not the N-terminus of the chemokine, and wherein said labeled chemokine is human CCL22, CCL2, CCL11, CCL19, CXCL12, CXCL11, CCL1, CCL18 or CXCL8.

22. The labeled chemokine of claim 18 wherein the label moiety is within the C-terminal half of the chemokine.

23. The labeled chemokine of any one of claims 18-22 which comprises a label moiety selected from the group consisting of biotin, avidin, streptavidin, fluorophore, lanthanide chelate, redox couple, paramagnetic group, chromophore, and radioactive labels.

24. A labeled polypeptide produced by the method of claim 1.

25. A method of detecting the presence of a receptor that binds to a labeled polypeptide of claim 24 comprising contacting said receptor with said labeled polypeptide and detecting the presence of said labeled polypeptide.

26. A method of detecting the presence of a chemokine receptor comprising contacting said receptor with a labeled chemokine according to any of claims 18-23 and detecting the presence of said labeled chemokine.

27. The method of claim 25 or 26 wherein the receptor is expressed on the surface of a cell.

28. The method of claim 25 or 26 wherein the presence of said receptor is indicative of a cellular subtype.

29. A method of screening for a modulator of the binding of a polypeptide to a receptor comprising the steps of contacting said receptor with a labeled polypeptide according to any of claims 18-24, in the presence and absence of a test compound, and detecting the relative amount of binding in the presence and absence of said test compound.

30. A method of screening for a modulator of receptor activation comprising the steps of contacting said receptor with a labeled polypeptide according to any of claims 18-24, in the presence and absence of a test compound, and detecting the relative amount of receptor activation in the presence and absence of said test compound.

31. The method of claim 29 or 30 wherein the test compound is an inhibitor of receptor binding or receptor activation.

32. A method of sorting cells, comprising the steps of contacting a labeled polypeptide according to any of claims 18-24 with said cells, and separating cells that bind said labeled polypeptide from cells that do not bind said labeled polypeptide.

33. The method of claim 32 wherein the method comprises use of fluorescent-activated cell sorting, magnetic beads, avidin-coupled beads, or streptavidin-coupled beads.

34. A medical imaging method comprising the steps of administering a labeled polypeptide according to any of claims 18-24 to a subject and detecting the location of said labeled polypeptide within the subject.
